(19) United States
(12) Patent Application Publication
(10) Pub. No.: US 2016/0222377 A1
(43) Pub. Date: Aug. 4, 2016

(54) METHOD FOR GENERATING ULTRA HIGH AFFINITY PEPTIDE LIGANDS

(71) Applicants: Terry T. Takahashi, Pasadena, CA (US); Richard W. Roberts, South Pasadena, CA (US)

(72) Inventors: Terry T. Takahashi, Pasadena, CA (US); Richard W. Roberts, South Pasadena, CA (US)

(21) Appl. No.: 15/009,721

(22) Filed: Jan. 28, 2016

Related U.S. Application Data
(60) Provisional application No. 62/109,583, filed on Jan. 29, 2015.

(51) Int. Cl. C12N 15/10; C07K 14/00

(52) U.S. Cl. CPC C12N 15/1062; C07K 14/00

(57) ABSTRACT

The present disclosure describes a method enabling the engineering of ligands with sub-nanomolar dissociation constants via a process of selection and extension (“extension selection”).

### CROSS REFERENCE TO RELATED APPLICATIONS

This application claims the benefit under 35 U.S.C. §119(e) of U.S. Provisional Application Ser. No. 62/109,583, filed Jan. 29, 2015, the contents of which is incorporated by reference in its entirety.

### STATEMENT OF GOVERNMENT SUPPORT

This invention was made with government support under Grant Nos. R21GM076678 and R01GM060416 awarded by National Institutes of Health (NIH). The government has certain rights in the invention.

### BACKGROUND

Peptide ligands are core to many life science applications, e.g., general research, therapeutics, diagnostics, drug discovery, purification protocols and for targeted delivery. Conventional techniques involving reiterative processes of creation and modification of polyclonal and monoclonal antibodies are time consuming and expensive. A need exits in the art to create high affinity peptide ligands that are selective and biologically relevant without a prolonged process of screening and modification. This invention satisfies this need and provides related advantages as well.

### SUMMARY

This disclosure provides a method for creating very high affinity peptides using an iterative process of selection followed by extension. The process has been used to create novel peptide compositions (sequences and sequence characteristics) that bind to the Bcl-xL protein with very high affinity.

The method described herein is useful for isolating ultra-high affinity peptide ligands with sub-nanomolar dissociation constants. Specifically, the method is a general methodology that enables engineering of relatively short peptides with nanomolar or picomolar or even better, binding affinities. Peptide reagents with high affinities are useful in multiple fields ranging from general research, therapeutics, diagnostics, for purification, or for targeted delivery.

The method described in this application has successfully created ligands for three proteins: Bcl-xL, streptavidin, and neutravidin. The peptides generated by this method exhibit very slow dissociation rates of less than 10⁻⁴ per second.

A method is provided for preparing a peptide ligand for biological activity. Non-limiting examples of biological activity include binding affinity of the peptide ligand for a target peptide; binding specificity to a target peptide; stability; resistance to degradation; and/or thermostability.

The method comprises, or alternatively consists essentially of, or yet further consists of, the steps of: a) obtaining an extension library comprising the step of linking an extension polynucleotide to a termini of each of a plurality of polynucleotides of the extension library, wherein each polynucleotide of the plurality encodes a pre-selected peptide ligand to a target peptide; b) translating the plurality of polynucleotides of the extension library to the corresponding peptide to obtain a peptide library; c) screening the peptide library for biological activity; and d) selecting the peptide ligand for the biological activity.

In one aspect, the steps can be repeated for each of the selected peptide ligands of step d) to obtain the desired biological activity. In one aspect, step a) through d) is repeated more than once.

In one aspect of the methods described herein, wherein termini of each of the plurality of polynucleotides of step a) is independently for each polynucleotide: the 5′ terminus, the 3′ terminus or both. In another aspect, the polynucleotides of the extension library comprise DNA and/or RNA.

Any suitable source can provide the plurality of polynucleotides, e.g., mRNA display; ribosome display; phage display; TRAP display; yeast display; selex; or peptide-on-plasmids. In one particular aspect, the plurality of polynucleotides is selected from an mRNA display library.

In one aspect, the extension library comprises only DNA polynucleotides. In these aspects, the method can be further modified by modifying the polynucleotides encoding the peptide library to facilitate linking the random polynucleotides to the termini of each of the polynucleotides. Non-limiting examples of such include PCR primer extension or restriction enzyme digestion.

In another aspect, the extension polynucleotide linked to the each of the plurality of polynucleotides encoding the target peptide library can be of any appropriate length or number, non-limiting examples of such include extension polynucleotides that comprise at least two nucleotide residues, or alternatively, between about 2 to about 150 nucleotides, and oligonucleotides there between, to comprise the extension polynucleotide linked to the each of the plurality of polynucleotides encoding the target peptide library. As is apparent to those of skill in the art, DNA is linked to DNA and RNA is linked to RNA. In a further aspect, the extension polynucleotide further comprises, or alternatively consists essentially of, or yet further consists of a spacer polynucleotide linked between the extension polynucleotide and the plurality of polynucleotides encoding the target peptide library. Non-limiting examples of such are shown in the figures (incorporated into the general disclosure herein) and for example, comprises the polynucleotide encoding the amino acid sequence Gly-Ser-Gly-Ser. Non-limiting examples of the length of the spacer polypeptide comprise between 1 and 100 amino acids, or alternatively between 2 and 100, or alternatively between 2 and 50, or alternatively between 2 and 40, or alternatively between 2 and 30, or alternatively between 2 and 25, or alternatively between 2 and 20, or alternatively between 2 and 15 or alternatively between 2 and 10, and variations there between.

The pre-selected peptide ligand may comprise an isolated naturally occurring polypeptide or alternatively one or more unnatural amino acids. Similarly, the plurality of polynucleotides and/or the extension polynucleotides may comprise an unnatural nucleotide.

The method may be further modified by linking to the selected peptide after step d) a detectable label, a cytotoxin or a therapeutic agent.

Further provided by this disclosure is an isolated peptide obtainable by a method as disclosed herein. Non-limiting examples of such include an isolated peptide comprising an sequence described herein and shown in any one of FIG. 4, FIG. 8, FIG. 10, FIG. 11, FIG. 12 and FIG. 13 (incorporated herein) as well as equivalents thereof. These peptides can further comprise, or alternatively consist essentially of, or yet further consist of, a detectable label, therapeutic agent or a cytotoxin. These can be combined with a carrier, e.g., a pharmaceutically acceptable carrier.

Isolated polynucleotides encoding the peptides of this disclosure are further provided. The polynucleotides can further comprise a gene expression vehicle as defined below, e.g., a plasmid, a liposome, a vector, for replication and/or expression and regulatory sequences operatively linked to them to facilitate expression and/or replication. These can be combined with a carrier, e.g., a pharmaceutically acceptable carrier.

Further provided are host cells an isolated peptide as described herein and/or an isolated polynucleotide as described herein. Host cells can be prokaryotic or eukaryotic, e.g, mammalian or human cells. They can be isolated from the mammal or cultured cell lines.

The peptides obtained and prepared by the methods of this disclosure can be used to target a cell or tissue expressing a target peptide, by contacting the cell or tissue with the isolated peptide as described herein. In one aspect, the peptides can be used to identify or purify a target ligand or a cell or bind to a cell expressing a target peptide by contacting the cell with the peptide of this disclosure.

In another aspect, the peptides identified and obtained by the methods disclosed herein have a therapeutic or diagnostic benefit or use. Thus, in an alternative aspect, a method is provided for treating or diagnosing a condition related to expression of a target peptide, comprising, or alternatively consisting essentially of, or yet further consisting of, administering an effective amount of the isolated peptide as described herein or a pharmaceutical composition containing such, to a subject in need thereof. In one aspect, the subject is a mammal, e.g., a equine, bovine, canine, feline or a human patient. In one aspect, the isolated peptide is administered by administration of a polynucleotide encoding the peptide.

Yet further disclosed is a kit for performing the method as disclosed herein comprising reagents to perform the methods and instructions for use.

### BRIEF DESCRIPTION OF THE DRAWINGS

FIG. 1 shows one aspect of the extension selection method of this disclosure. An initial library (here X₉4, where X=all 20 amino acids and 4=an NTG codon coding for either Met (ATG), Leu (CTG or TTG), or Val (GTG)) is selected for binding to the target (here, Bcl-xL). After several rounds of selection, an enriched DNA library is generated. Random sequence (shown in red (color scale or gray in black or white scale)) is then ligated to either the 5′ or 3′ end of the enriched library, to create an N- or C-terminal extended library, respectively. This extended library is then used for further selection against the target.

FIGS. 2A-2E show A) PCR with a 5′ primer that encodes an AcuI site introduces an AcuI site in the DNA library. B) Digest of the PCR product from A results in an AcuI fragment containing a 5′-CA-3′ overhang. C) PCR with a 3′ primer that encodes a BpmI site introduces allows D) digestion of the library with BpmI, creating a BpmI fragment containing a 5′-TG-3′ overhang. E) The AcuI fragment and BpmI fragments are combined and ligated with T4 DNA ligase.

FIG. 3 shows in vitro selection of the first library (X₉4) against Bcl-xL target. Bcl-xL was biotinylated and immobilized on neutravidin agarose and the first library was screened (or selected for) binding against Bcl-xL using mRNA display. Using mRNA display, radiolabeled peptides were synthesized using ³⁵S-labeled methionine and attached to their encoding mRNA. These mRNA-peptide fusions were then incubated with Bcl-xL/neutravidin beads (blue (color scale, dark gray (black and white scale)); labeled Bcl-xL) or against neutravidin beads alone (red color scale, light gray (black and white scale)); no target). The beads were washed, and the remaining radioactivity counted in a scintiallation counter. The percent bound was calculated by taking the number of cpm on the beads divided by the total cpm of the reaction (flow through, washes, and beads).

FIG. 4 shows sequences from the Round 5 selection against Bcl-xL. Clones in blue (color scale, gray (black and white scale)) have been tested for binding. Periods (.) represent stop codons. Lower case letters represent amino acids coded by the 3′ primer used for amplification in mRNA display.

FIG. 5 shows percent binding of the sequences from the Round 5 first selection. Radiolabeled mRNA display fusions of each sequence were tested for binding against Bcl-xL immobilized on neutravidin agarose (Blue bars (color scale, dark gray (black and white scale)); +Bcl-xL) or against neturavidin agarose only (red bars (color scale, light gray (black and white scale)); no target). mRNA display fusions were treated with RNase to degrade the mRNA, and also tested for binding against immobilized Bcl-xL (green (color scale, gray (black and white scale)); +Bcl-xL, +RNase).

FIGS. 6A-6B show A) percent binding of the N-terminally extended library against Bcl-xL. B) Percent binding of the C-terminally extended library against Bcl-xL.

FIG. 7 shows a test to show that the binding of peptides from the N- or C-extended libraries (NExt or CExt) do not depend on the presence of reducing agent, and do not form disulfide bonds with Bcl-xL protein. Both N- and C- extended libraries were tested for binding in the presence (+DTT) or absence (No DTT) of DTT. No significant difference is seen, showing that disulfide bond formation between the peptides and target do not occur.

FIG. 8 shows sequences from both N- and C-terminal extended libraries. A core binding sequence is seen in all cases. The difference between family 1A, 1B, and 1C is in the last 6 amino acids.

FIG. 9 shows percent binding of clones CExt7-5 or CExt7-11 tested using radiolabeled mRNA display fusions of each clone. Binding was tested against neutravidin beads only (red; no target) or Bcl-xL (blue; +Bcl-xL). Fusions were treated with RNase (green; +Bcl-xL, +RNase) to show that mRNA was not the species responsible for binding.

FIG. 10 shows sequence alignment with known Bcl-xL binding peptides.

FIG. 11 shows off rates of CExt7C-5 and CExt7C-11. Radiolabeled mRNA displayed fusions were bound to immobilized Bcl-xL, washed and non-biotinylated Bcl-xL was added as a competitor at a 100-fold molar excess, to approximate pseudo-first order conditions. Aliquots were taken at various time points and the percent of cpm remaining on the beads determined. The off-rates were fit with using a single exponential and correspond to 6×10⁻⁶ and 2×10⁻⁵ per second for CExt7C-5 and CExt7C-11, respectively. Assuming typically macromolecular association constants of 10⁵ M⁻¹s⁻¹, this corresponds to K_{D}s of 60 and 200 pM at room temperature for CExt7C-5 and CExt7C-11, respectively.

FIG. 12 shows sequences from Round 4 of the BclDoped selection.

FIG. 13 shows doped sequences from Round 4 obtained by Illumina sequencing.

FIG. 14 shows off rate for clone BclDoped 4.10 compared to the off rate of the parental CExt7C-5 sequence. The doped clone (4.10) has an off rate of 2.4E-6/s.

### DETAILED DESCRIPTION

Unless defined otherwise, all technical and scientific terms used herein have the same meanings as commonly understood by one of ordinary skill in the art to which this invention belongs. All nucleotide sequences provided herein are presented in the 5′ to 3′ direction. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/− 15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporidae, such as rabbits, livestock, sport animals, and pets.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence.

As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

The phrase “equivalent polypeptide” or “equivalent polynucleotide” refers to protein, polynucleotide, or peptide fragment which hybridizes to the exemplified polynucleotide or polypeptide encoding such or its complement, under stringent conditions and which exhibit similar biological activity in vitro or in vivo, e.g., approximately 100%, or alternatively, over 90% or alternatively over 85% or alternatively over 70%, as compared to the standard or control biological activity. Additional embodiments of “equivalents” are identified by sequence identity to the reference polypeptide or polynucleotide, e.g., having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology or identity. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW (available at the web address://align.genome.jp/, last accessed on Mar. 7, 2011.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6× SSC to about 10× SSC; formamide concentrations of about 0% to about 25%; and wash solutions from about 4× SSC to about 8× SSC. Examples of moderate hybridization conditions include: incubation temperatures of about 40° C. to about 50° C.; buffer concentrations of about 9× SSC to about 2× SSC; formamide concentrations of about 30% to about 50%; and wash solutions of about 5× SSC to about 2× SSC. Examples of high stringency conditions include: incubation temperatures of about 55° C. to about 68° C.; buffer concentrations of about 1× SSC to about 0.1× SSC; formamide concentrations of about 55% to about 75%; and wash solutions of about 1× SSC, 0.1× SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “stop codon” intends a three nucleotide contiguous sequence within messenger RNA that signals a termination of translation. Non-limiting examples include in RNA, UAG, UAA, UGA and in DNA TAG, TAA or TGA. Unless otherwise noted, the term also includes nonsense mutations within DNA or RNA that introduce a premature stop codon, causing any resulting protein to be abnormally shortened. For example, one can remove all the nucleotides encoding for valine, and then reduce in positions where they are not typically located.

As used herein, the term “stable peptide” intends a peptide, polypeptide or protein that has a lifetime, once administered in vivo, which is sufficient to reach target cells and to exert its biological action. These peptides have a conformation which protect them against degradation by cell proteases while retaining biological activity. An indication of the stability of a peptide may be obtained using tests carried out in vitro. For example, in vitro degradation of a peptide is measured by contact with a variety of purified proteases, which are commercially available, for increasing incubation periods (1 hour to 72 hours, for example). Peptide degradation is then demonstrated by reverse phase HPLC, comparing the profiles obtained before and after digestion. In one aspect, a stable protein is more resistant to proteases present in human serum, e.g., more than about 20%, or alternatively, more than about 40%, or alternatively more than about 50%, or alternatively more than about 60%, or alternatively than about 70%, or alternatively more than about 75%, or alternatively more than 80% more resistant.

As used herein, the term “mRNA library” intends a plurality of at least two RNA members. Similarly, a “polynucleotide library” intends a plurality of at least two polynucleotides (DNA, RNA or both) members.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this invention can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., PCT Publication No. WO 95/27071. Ads do not require integration into the host cell genome. Recombinant Ad derived vectors, particularly those that reduce the potential for recombination and generation of wild-type virus, have also been constructed. See, PCT Publication Nos. WO 95/00655 and WO 95/11984. Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. DNA virus, RNA virus, modifications, liposomes are non-limiting examples of vectors.

Gene delivery vehicles also include DNA/liposome complexes, micelles and targeted viral protein-DNA complexes. Liposomes that also comprise a targeting antibody or fragment thereof can be used in the methods of this invention. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues.

As used herein, the term “label” intends a directly or indirectly detectable compound or composition that is conjugated directly or indirectly to the composition to be detected, e.g., N-terminal histadine tags (N-His), magnetically active isotopes, e.g., ¹¹⁵-Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition, which is detectable. The labels can be suitable for small-scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Detectable labels include fluorescent labels. Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The terms “antigen” and “antigenic” refer to molecules with the capacity to be recognized by an antibody or otherwise act as a member of an antibody-ligand pair. “Specific binding” refers to the interaction of an antigen with the variable regions of immunoglobulin heavy and light chains. Antibody-antigen binding may occur in vivo or in vitro. The skilled artisan will understand that macromolecules, including proteins, nucleic acids, fatty acids, lipids, lipopolysaccharides and polysaccharides have the potential to act as an antigen. The skilled artisan will further understand that nucleic acids encoding a protein with the potential to act as an antibody ligand necessarily encode an antigen. The artisan will further understand that antigens are not limited to full-length molecules, but can also include partial molecules. The term “antigenic” is an adjectival reference to molecules having the properties of an antigen. The term encompasses substances which are immunogenic, i.e., immunogens, as well as substances which induce immunological unresponsiveness, or anergy, i.e., anergens.

### MODES FOR CARRYING OUT THE DISCLOSURE

### Polypeptides

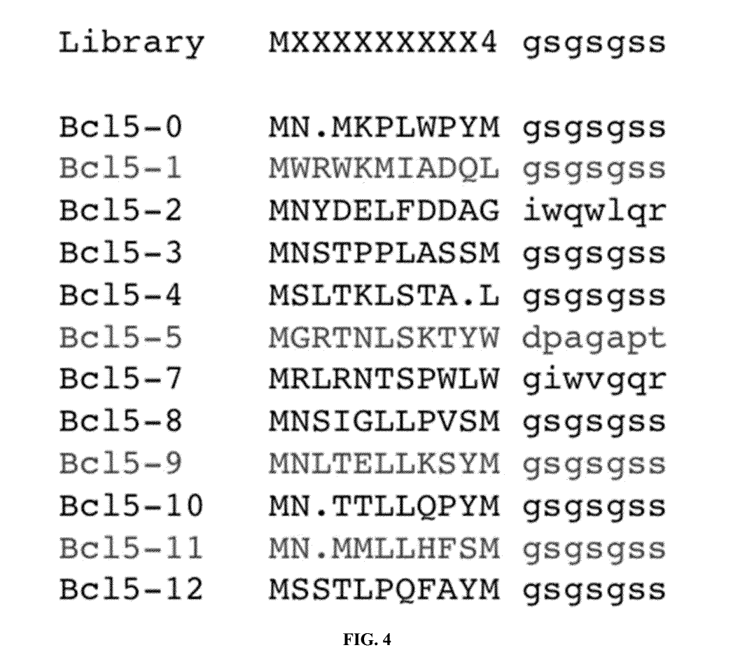

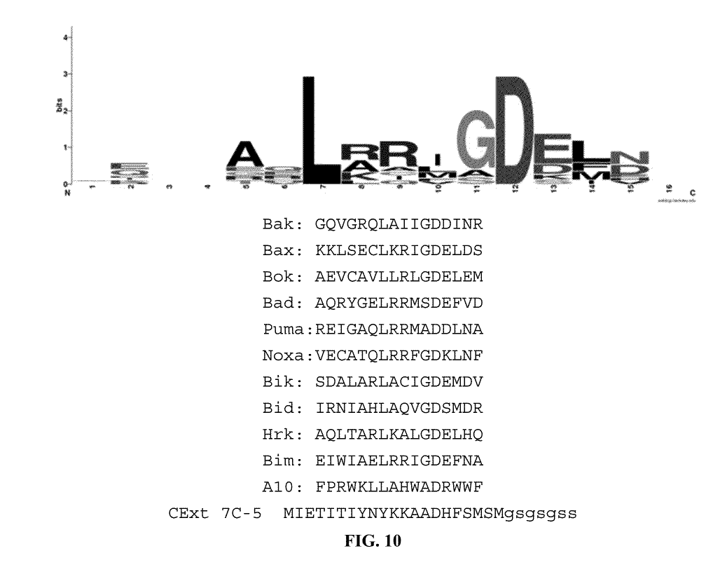

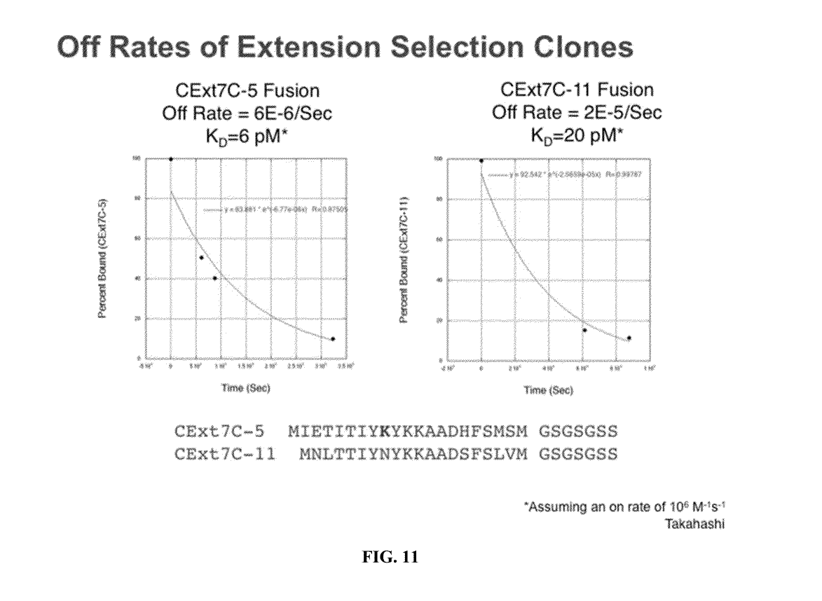

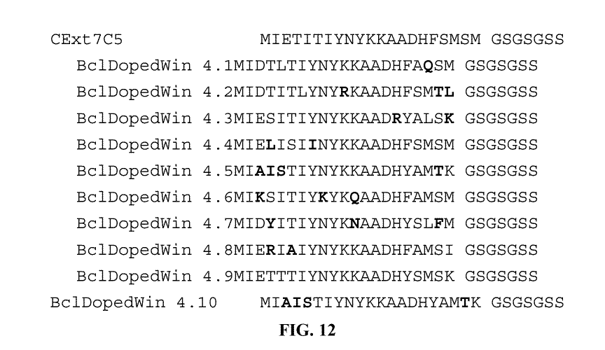

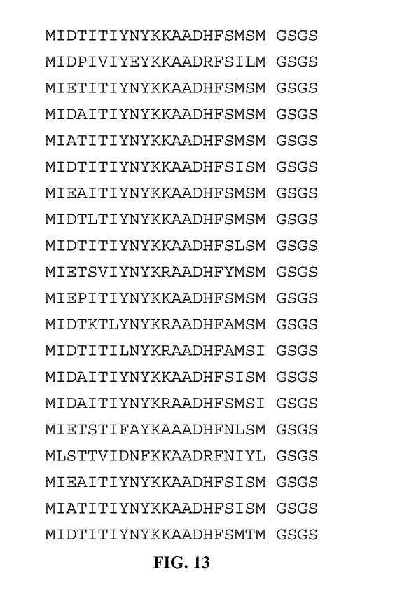

Using the methods described herein, novel polypeptides having enhanced diagnostic and therapeutic utility can be efficiently produced and screened. Non-limiting examples of such include an isolated peptide comprising a sequence described herein and shown in any one of FIG. 4, FIG. 8, FIG. 10, FIG. 11, FIG. 12 and FIG. 13 (incorporated herein) as well as equivalents thereof. In another aspect, the polypeptides of this disclosure comprise the below noted polypeptides and equivalents of each thereof:
Met at Position 1—
Ile, Cys, Asp, Phe, Gly, Leu, Met, Asn, Pro, Gln, Ser, Thr, or Val at Position 2
Asp, Ala, Glu, Phe, Gly, His, Ile, Lys, Leu, Met, Asn, Pro, Gln, Arg, Ser, Thr, Val, Trp, or Tyr at Position 3
Any amino acid at Position 4
Any Amino acid at Position 5
Thr, Ala, Phe, His, Ile, Lys, Leu, Met, Gln, Arg, Ser, Val, Trp, or Tyr at Position 6
Ile, Leu, Met, Asn, Pro, Arg, or Val at Position 7
Tyr, Ala, Cys, Asp, Glu, Phe, Gly, His, Ile, Lys, Leu, Met, Asn, Gln, Arg, Ser, Thr, Val, or Trp at Position 8
Asn, Ala, Cys, Asp, Glu, Phe, His, Ile, Lys, Leu, Met, Gln, Arg, Ser, Thr, Val, or Tyr at Position 9
Tyr, Phe, Lys, Arg, Ser, or Trp at Position 10
Lys, Ile, Leu, Met, Gln, Arg, or Val at Position 11
Lys, Ala, Cys, Asp, Glu, Gly, His, Ile, Lys, Leu, Met, Asn, Gln, Arg, Ser, Thr, Val, Trp, or Tyr at Position 12
Ala, Asp, Phe, Ile, Pro, Ser, or Thr at Position 13
Ala, Leu, Met, Pro, or Ser at Position 14
Asp, Ala, or Pro at Position 15
His, Ala, Cys, Asp, Glu, Leu, Asn, Gln, Arg, Ser, Thr, Trp, or Tyr at Position 16
Phe, Ala, His, Asn, Pro, or Tyr at Position 17
Ser, Ala, Asp, Phe, Gly, His, Leu, Asn, Gln, Thr, Trp, or Tyr at Position 18
Met, Ala, Cys, Asp, Glu, Phe, Gly, His, Ile, Lys, Leu, Asn, Gln, Arg, Ser, Thr, Val, Trp, or Tyr at Position 19
Any Amino acid at Position 20
Met, Phe, His, Ile, Lys, Leu, Pro, Gln, Arg, Ser, Val, or Trp at Position 21

These peptides can further comprise, or alternatively consist essentially of, or yet further consist of, a detectable label, therapeutic agent or a cytotoxin covalently attached to the polypeptide. The polypeptides can be combined with a carrier, e.g., a pharmaceutically acceptable carrier. Also provided herein are the polynucleotides encoding the polypeptides, alone or in combination with expression vectors, labels and host cells for recombinant production.

After selection by the method described herein, reproduction and expression of a polynucleotide to obtain the polypeptides are obtainable by a number of processes known to those of skill in the art, which include purification, chemical synthesis and recombinant methods. Polypeptides can be isolated from preparations such as host cell systems by methods such as immunoprecipitation with antibody, and standard techniques such as gel filtration, ion-exchange, reversed-phase, and affinity chromatography. For such methodology, see for example Deutscher et al. (1999) Guide To Protein Purification: Methods In Enzymology (Vol. 182, Academic Press). Accordingly, this disclosure also provides the processes for obtaining these polypeptides as well as the products obtainable and obtained by these processes.

The polypeptides also can be reproduced by chemical synthesis using a commercially available automated peptide synthesizer such as those manufactured by Perkin/ Elmer/Applied Biosystems, Inc., Model 430A or 431A, Foster City, Calif., USA. The synthesized polypeptide can be precipitated and further purified, for example by high performance liquid chromatography (HPLC). Accordingly, this disclosure also provides a process for chemically synthesizing the polypeptides of this disclosure by providing the sequence of the protein and reagents, such as amino acids and enzymes and linking together the amino acids in the proper orientation and linear sequence.

Also provided by this disclosure are the peptides described herein conjugated to a detectable agent for use in therapeutic or diagnostic methods. For example, detectably labeled peptides can be bound to a column and used for the detection and purification of antibodies. They also are useful as immunogens for the production of antibodies. The peptides of this disclosure are useful in an in vitro assay system to screen for agents or drugs, which modulate cellular processes. For example, detectably labeled peptides can be bound to a column and used for the detection and purification of antibodies. The polypeptides can have therapeutic use by administration of an effective amount of the polypeptide to a subject (animal, mammal, human, canine, feline or equine, for example) to treat the disease or condition. Accordingly, this disclosure also provides the antibodies that specifically bind to the polypeptides of this disclosure. The antibodies are generated using techniques know in the art.

It is well known to those skilled in the art that modifications can be made to the peptides of the disclosure to provide them with altered properties.

Peptides of the disclosure can be modified to include unnatural amino acids. Thus, the peptides may comprise D-amino acids, a combination of D- and L-amino acids, and various “designer” amino acids (e.g., .beta.-methyl amino acids, C-.alpha.-methyl amino acids, and N-.alpha.-methyl amino acids, etc.) to convey special properties to peptides. Additionally, by assigning specific amino acids at specific coupling steps, peptides with .alpha.-helices .beta. turns, .beta. sheets, .gamma.-turns, and cyclic peptides can be generated. Generally, it is believed that .alpha.-helical secondary structure or random secondary structure is preferred.

The peptides of this disclosure also can be combined with various solid phase carriers, such as an implant, a stent, a paste, a gel, a dental implant, or a medical implant or liquid phase carriers, such as beads, sterile or aqueous solutions, pharmaceutically acceptable carriers, pharmaceutically acceptable polymers, liposomes, micelles, suspensions and emulsions. Examples of non-aqueous solvents include propyl ethylene glycol, polyethylene glycol and vegetable oils.

The peptides of this invention can further comprise a carrier such as a pharmaceutically acceptable carrier. In one aspect, the carrier is one that is suitable for oral administration.

In one aspect, the peptides are useful therapeutically, comprising administering to the subject an effective amount of a suitable peptide, polypeptide, polynucleotide, or composition of this disclosure. In one aspect, the subject is a human patient.

### Antibodies

The disclosure, in another aspect, provides an antibody that binds an isolated polypeptide of the disclosure. The antibody can be a polyclonal antibody, a monoclonal antibody, a chimeric antibody, a humanized antibody or a derivative or fragment thereof as defined below. In one aspect, the antibody is detectably labeled or further comprises a detectable label conjugated to it.

Also provided is a composition comprising the antibody and a carrier. Further provided is a biologically active fragment of the antibody, or a composition comprising the antibody fragment. Suitable carriers are defined supra.

Further provided is an antibody-peptide complex comprising, or alternatively consisting essentially of, or yet alternatively consisting of, the antibody and a polypeptide specifically bound to the antibody. In one aspect, the polypeptide is the polypeptide against which the antibody is raised.

This disclosure also provides an antibody capable of specifically forming a complex with a protein or polypeptide of this disclosure, which are useful in the therapeutic methods of this disclosure. The term “antibody” includes polyclonal antibodies and monoclonal antibodies, antibody fragments, as well as derivatives thereof (described above). The antibodies include, but are not limited to mouse, rat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify therapeutic polypeptides.

### Polynucleotides

This disclosure also provides isolated or recombinant polynucleotides encoding one or more of the above-identified peptides and their respective complementary strands. Gene delivery vehicles such as vectors comprising the isolated or recombinant polynucleotides are further provided examples of which are known in the art and briefly described herein. In one aspect where more than one isolated or recombinant polynucleotide is to be expressed as a single unit, the isolated or recombinant polynucleotides can be contained within a polycistronic vector. The polynucleotides can be DNA, RNA, mRNA or interfering RNA, such as siRNA, miRNA or dsRNA.

The disclosure further provides the isolated or recombinant polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)) which contains maps, functional properties, commercial suppliers and a reference to GenEMBL accession numbers for various suitable vectors.

In one embodiment, the polynucleotides of the disclosure encode polypeptides or proteins having diagnostic and therapeutic utilities as described herein as well as probes to identify transcripts of the protein that may or may not be present.

Expression vectors containing these nucleic acids are useful to obtain host vector systems to produce proteins and polypeptides. It is implied that these expression vectors must be replicable in the host organisms either as episomes or as an integral part of the chromosomal DNA. Non-limiting examples of suitable expression vectors include plasmids, yeast vectors, viral vectors and liposomes. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using known methods. See Sambrook, et al. (1989) supra. In addition to the use of viral vector for insertion of exogenous nucleic acid into cells, the nucleic acid can be inserted into the host cell by methods known in the art such as transformation for bacterial cells; transfection using calcium phosphate precipitation for mammalian cells; or DEAE-dextran; electroporation; or microinjection. See, Sambrook et al. (1989) supra, for methodology. Thus, this disclosure also provides a host cell, e.g. a mammalian cell, an animal cell (rat or mouse), a human cell, or a prokaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this disclosure can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller et al. (1989) BioTechniques 7:980-990). The methodology of using replication-incompetent retroviruses for retroviral-mediated gene transfer of gene markers has been established. (Bordignon (1989) PNAS USA 86:8912-8952; Culver (1991) PNAS USA 88:3155; and Rill (1991) Blood 79(10):2694-2700).

This disclosure also provides genetically modified cells that contain and/or express the polynucleotides or polypeptides of this disclosure. The genetically modified cells can be produced by insertion of upstream regulatory sequences such as promoters or gene activators (see, U.S. Pat. No. 5,733,761), or by insertion of the peptides of this disclosure.

The polynucleotides also can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this disclosure further provides a method for detecting a single-stranded polynucleotide or its complement, by contacting target single-stranded polynucleotide with a labeled, single-stranded polynucleotide (a probe) which is a portion of the polynucleotide of this disclosure under conditions permitting hybridization (preferably moderately stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

The polynucleotide embodied in this disclosure can be obtained using chemical synthesis, recombinant cloning methods, PCR, or any combination thereof. Methods of chemical polynucleotide synthesis are known in the art and need not be described in detail herein. One of skill in the art can use the sequence data provided herein to obtain a desired polynucleotide by employing a DNA synthesizer or ordering from a commercial service.

One method to amplify polynucleotides is PCR and kits for PCR amplification are commercially available. After amplification, the resulting DNA fragments can be detected by any appropriate method known in the art, e.g., by agarose gel electrophoresis followed by visualization with ethidium bromide staining and ultraviolet illumination.

As noted above, the polynucleotides of this disclosure can be isolated or replicated using PCR. The PCR technology is the subject matter of U.S. Pat. Nos. 4,683,195; 4,800,159; 4,754,065; and 4,683,202 and described in PCR: The Polymerase Chain Reaction (Mullis et al. eds., Birkhauser Press, Boston (1994)) or MacPherson et al. (1991) and (1995) supra, and references cited therein. Alternatively, one of skill in the art can use the sequences provided herein and a commercial DNA synthesizer to replicate the DNA. Accordingly, this disclosure also provides a process for obtaining the polynucleotides of this disclosure by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods known to those of skill in the art. A process for obtaining polynucleotides by this method is further provided herein as well as the polynucleotides so obtained.

Alternatively, RNA can be obtained by first inserting a DNA polynucleotide into a suitable host cell. The DNA can be delivered by any appropriate method, e.g., by the use of an appropriate gene delivery vehicle (e.g., liposome, plasmid or vector) or by electroporation. When the cell replicates and the DNA is transcribed into RNA; the RNA can then be isolated using methods known to those of skill in the art, for example, as set forth in Sambrook et al. (1989) supra. For instance, mRNA can be isolated using various lytic enzymes or chemical solutions according to the procedures set forth in Sambrook et al. (1989) supra, or extracted by nucleic-acid-binding resins following the accompanying instructions provided by manufactures.

Polynucleotides exhibiting sequence complementarity or homology to a polynucleotide of this disclosure are useful as hybridization probes or as an equivalent of the specific polynucleotides identified herein. Since the full coding sequence of the transcript is known, any portion of this sequence or homologous sequences, can be used in the methods of this disclosure.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA is at least about 80% identical to the homologous region. More preferably, the probe is 85% identical to the corresponding gene sequence after alignment of the homologous region; even more preferably, it exhibits 90% identity.

These probes can be used in radioassays (e.g. Southern and Northern blot analysis) to detect, prognose, diagnose or monitor various cells or tissues containing these cells. The probes also can be attached to a solid support or an array such as a chip for use in high throughput screening assays for the detection of expression of the gene corresponding a polynucleotide of this disclosure. Accordingly, this disclosure also provides a probe comprising or corresponding to a polynucleotide of this disclosure, or its equivalent, or its complement, or a fragment thereof, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally preferred, so as to increase stability and selectivity of the hybrid, and thereby improving the specificity of particular hybrid molecules obtained. More preferably, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. In one aspect, a probe is about 50-75 or more alternatively, 50-100, nucleotides in length.

The polynucleotides of the present disclosure can serve as primers for the detection of genes or gene transcripts that are expressed in cells described herein. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. For illustration purposes only, a primer is the same length as that identified for probes.

Methods for administering an effective amount of a gene delivery vector or vehicle to a cell have been developed and are known to those skilled in the art and described herein. Methods for detecting gene expression in a cell are known in the art and include techniques such as in hybridization to DNA microarrays, in situ hybridization, PCR, RNase protection assays and Northern blot analysis. Such methods are useful to detect and quantify expression of the gene in a cell. Alternatively expression of the encoded polypeptide can be detected by various methods. In particular it is useful to prepare polyclonal or monoclonal antibodies that are specifically reactive with the target polypeptide. Such antibodies are useful for visualizing cells that express the polypeptide using techniques such as immunohistology, ELISA, and Western blotting. These techniques can be used to determine expression level of the expressed polynucleotide.

### Compositions

Compositions are further provided. The compositions comprise a carrier and one or more of an isolated polynucleotide of the disclosure, an isolated polypeptide of the disclosure, an antibody, a gene delivery vehicle of the disclosure or an isolated host cell of the disclosure. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of an isolated polypeptide of the disclosure, an isolated polynucleotide of the disclosure, a vector of the disclosure, an isolated host cell of the disclosure, or an antibody of the disclosure, formulated with one or more pharmaceutically acceptable auxiliary substances.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of an isolated peptide of the disclosure, an isolated polynucleotide of the disclosure, a vector of the disclosure, an isolated host cell of the disclosure, can be formulated into preparations for administration in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other anticancer agents. For intravenous administration, suitable carriers include physiological saline, or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations of the disclosure comprise a peptide of the disclosure formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself (e.g. by an elastic bladder)).

Suppositories of the disclosure can be prepared by mixing a compound of the disclosure with any of a variety of bases such as emulsifying bases or water-soluble bases. Embodiments of this pharmaceutical formulation of a compound of the disclosure can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds of the disclosure. Similarly, unit dosage forms for injection or intravenous administration may comprise a compound of the disclosure in a composition as a solution in sterile water, normal saline or another pharmaceutically acceptable carrier.

Embodiments of the pharmaceutical formulations of the disclosure include those in which one or more of an isolated polypeptide of the disclosure, an isolated polynucleotide of the disclosure, a vector of the disclosure, an isolated host cell of the disclosure, or an antibody of the disclosure is formulated in an injectable composition. Injectable pharmaceutical formulations of the disclosure are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the disclosure.

In an embodiment, one or more of an isolated polypeptide of the disclosure, an isolated polynucleotide of the disclosure, a gene delivery vehicle or vector of the disclosure, or an isolated host cell of the disclosure is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound of the disclosure can be accomplished using any of a variety of refillable, pump systems. Pumps provide consistent, controlled release over time. In some embodiments, a compound of the disclosure is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a peptide of the disclosure are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. After administration, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the peptide (as well as combination compositions) is delivered in a controlled release system. For example, a peptide of the disclosure may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose.

In another embodiment, the compositions of the present disclosure (as well as combination compositions separately or together) include those formed by impregnation of a peptide described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Therapeutic Methods

Further provided are methods for treating a subject in need of treatment, comprising consisting essentially of, or yet further consisting of, administering to the subject an effective amount of one or more of a polypeptide, an antibody, and/or polynucleotide and/or host cell obtainable by the methods of this disclosure, or a composition of this disclosure, or a combination of any thereof.

### Combination Therapy

The compositions and related methods of the present disclosure may be used in combination with the administration of other therapies as appropriate. The additional therapeutic treatment can be added prior to, concurrent with, or subsequent to methods or compositions described herein, and can be contained within the same formulation or as a separate formulation.

### Screening Assays

The present disclosure provides methods for screening for equivalent agents, such as equivalent peptides to a peptide or composition of this disclosure, and various agents that modulate the activity of the active agents and pharmaceutical compositions of the disclosure or the function of a polypeptide or peptide product encoded by the polynucleotide of this disclosure. For the purposes of this disclosure, an “candidate agent” is intended to include, but not be limited to a biological or chemical compound such as a simple or complex organic or inorganic molecule, a peptide, a protein (e.g. antibody), a polynucleotide (e.g. anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

### Kits

Kits containing the agents and instructions necessary to perform the in vitro and in vivo methods as described herein also are claimed. Accordingly, the disclosure provides kits for performing these methods which may include peptides and/or other composition of this disclosure as well as instructions for carrying out the methods of this disclosure such as collecting tissue and/or performing the screen, and/or analyzing the results, and/or administration of an effective amount of a peptide or other composition as described herein. These can be combined with other known or other candidate agents.

### Peptide Libraries and Method

The disclosed method involves generating or obtaining two or more ligands against a target of interest. For example, if more than one ligand is known in the literature to bind a target of interest, these ligands can serve as the starting point for this extension selection method. Alternatively, many methods are known to generate novel ligands against a target of interest including, but not limited to, mRNA display (Roberts and Szostak (1997) Proc Natl Acad Sci USA 94: 12297), ribosome display (Hanes and Pluckthun (1997) Proc Natl Acad Sci USA 94: 4937), phage display (Smith and Petrenko (1997) Chem Rev 97: 391), TRAP display (Ishizawa, et al. (2013) J Am Chem Soc 135: 5433), yeast display (Boder and Wittrup (1997) Nat Biotechnol 15: 553), selex (Tuerk and Gold (1990) Science 249: 505), or peptide-on-plasmids (Cull, et al. (1992) Proc Natl Acad Sci USA 89: 1865). Most preferably, the method used for generating these ligands is mRNA display. Two or more ligands generated by one of these selection methods can also serve as a starting point for the extension selection method.

Once two or more ligands are obtained, then random sequence is added to either end or both of the ligand, thus extending the length of the initial ligands. In the case of a peptide, the random sequence can either be added to the N-terminus or the C-terminus. In some embodiments, the random sequence can be added to both the N- and C-terminus simultaneously. By adding the random sequence to either or both ends of the initial ligand, extended libraries are created.

The random sequence can be added to the initial ligands by a variety of methods known in the art. Typically, random peptide libraries are created by first constructing DNA or mRNA that codes for the random library, and then translating the nucleic acid to create the random peptide library. Thus, the extended libraries can be created by adding a randomized nucleic acids to the polynucleotide encoding the initial ligands. For example, an extended RNA library can be created using two or more RNAs that code for the initial ligands and then ligating random mRNA sequence to the 3′ end of the initial RNA ligands using an RNA ligase (for example, T4 RNA ligase). In some embodiments, an extended RNA library can be translated to create an extended peptide library.

More preferably, the extended libraries are created at the DNA level. In this case, an extended DNA library can be created by taking two or more DNA molecules that code for the initial ligand or ligand library, and ligating random DNA to either the 5′ or 3′ end (or both) of the initial DNA. In a preferred embodiment, the DNA is first digested with a restriction enzyme that allows more efficient ligation between the two DNA fragments. More preferably, the restriction enzyme that is used is a Type IIS enzyme, which cleaves at a distance from the restriction enzyme binding site. In a preferred embodiment, the Type IIS enzymes are AcuI and BpmI. In another embodiment, the Type IIS enzyme is BciIV.

The number of random residues added to the end of the ligand is more than 2 amino acids, and can be up to 100 amino acids residues. Most preferably, the number of residues is between 6 and 9 amino acids for a peptide ligand. As is apparent to the skilled artisan, when the library is a polynucleotide, polynucleotides are added in the appropriate number to the polynucleotides of the library. In some embodiments, there is a spacer of constant sequence between the initial ligand and the random sequence. For example, for a peptide ligand a spacer could be the peptide comprising Gly—Ser—Gly—Ser. The spacer could have a length of one or more amino acid residues, up to 100 amino acid residues (or the polynucleotides encoding them in the case of polynucleotide libraries). In a preferred embodiment, there is no spacer between the initial ligand and the random sequence.

These extended libraries are then used in a second in vitro selection to obtain a second set of ligands with improved function relative to the starting ligands. The improved function could be binding affinity to a target, improved binding specificity for a target, or another property that can be improved by an in vitro selection, for example, resistance to degradation or thermostability.

The second set of ligands can then be used for the desired purpose, or, if these ligands are still suboptimal, the extension selection method can be repeated. The process can be repeated until the ligands have the desired level of fitness.

In one embodiment of this disclosure, the ligands are composed of natural residues: any of the 20 natural amino acids for proteins/peptides or any of the four nucleotides in RNA. In another embodiment, the ligands contain one or more unnatural residues. For example, a peptide could contain one or more of N-methyl amino acids, C-alpha methyl amino acids, Beta amino acids, D-amino acids, or Peptide-nucleic acid. An RNA sequence could contain unnatural bases such as a 2′-OMe, 2′-F, or 2′-NH₂.

### EXAMPLES

The following examples are intended to illustrate, and not limit, the disclosures disclosed herein.

### Example 1

### Generation of Sub-Nanomolar Ligands Against Bcl-xL

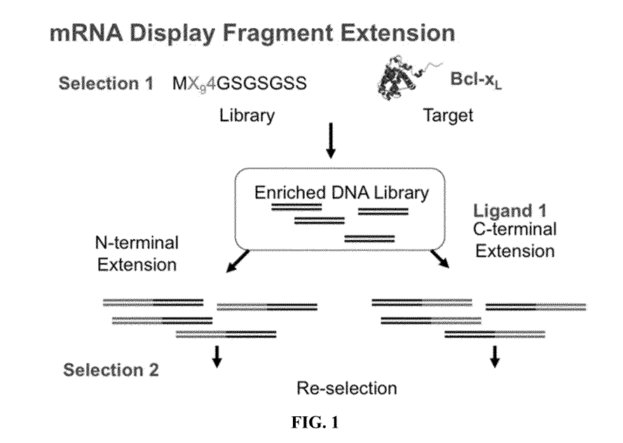

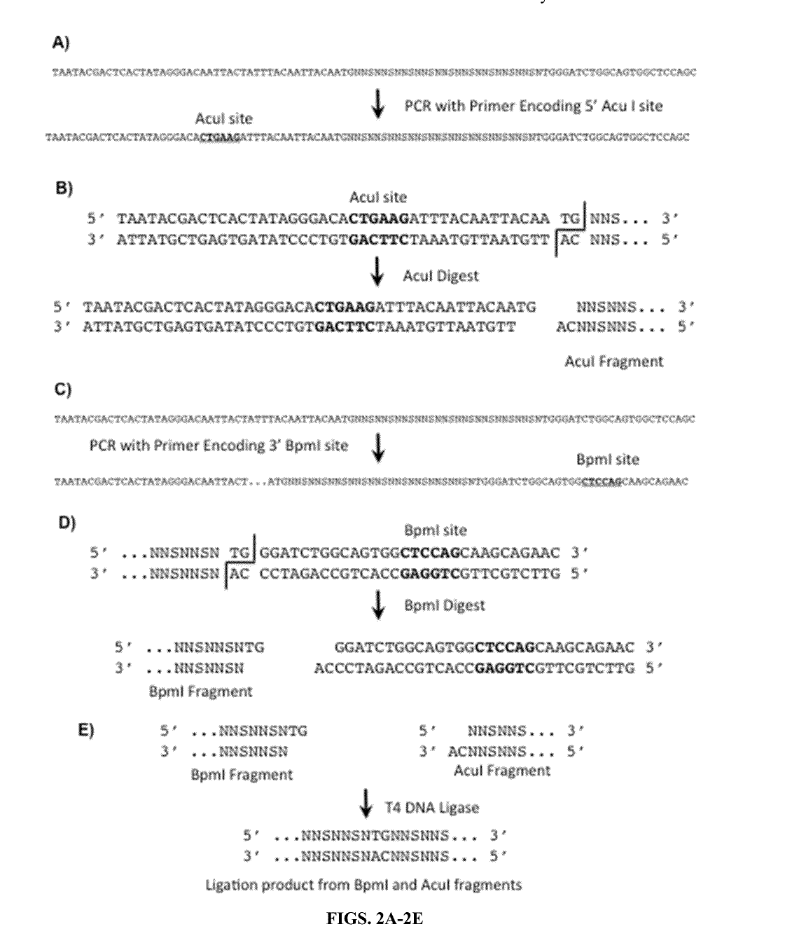

First, a peptide library was designed. The library was:
Met-X₉-4-gsgsgss
   where X represents any of the 20 natural amino acids, 4 is an NTG codon (where N=T, C, A, or G; this codon codes for Met (ATG), Leu (CTG or TTG), or Val (GTG)), and gsgsgss is a Gly (G) and Ser (S) spacer that is used for PCR amplification of the library. The NTG codon was designed in order to provide a constant sequence for extending the initial library (FIG. 1 and FIG. 2).

A initial peptide was then generated using mRNA display. To do this, the DNA for this library was synthesized, PCR amplified, and transcribed into mRNA. A synthetic DNA linker containing a 3′ puromycin (pF30P) was ligated to the 3′ end of the mRNA, and the ligation product was purified. The ligation product was then translated in vitro using rabbit reticulocyte lysate to generate an mRNA display library of peptides, each of which was fused to its encoding mRNA. These mRNA peptide fusions were then purified, reverse transcribed, and selected for binding against immobilized target. The bound cDNA was then PCR amplified, and the process repeated.

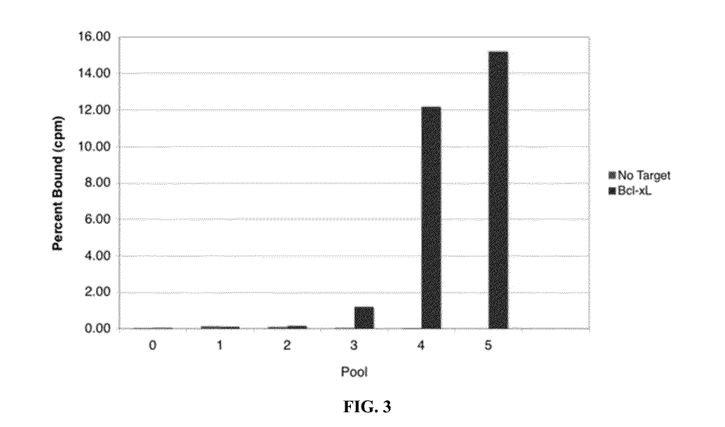

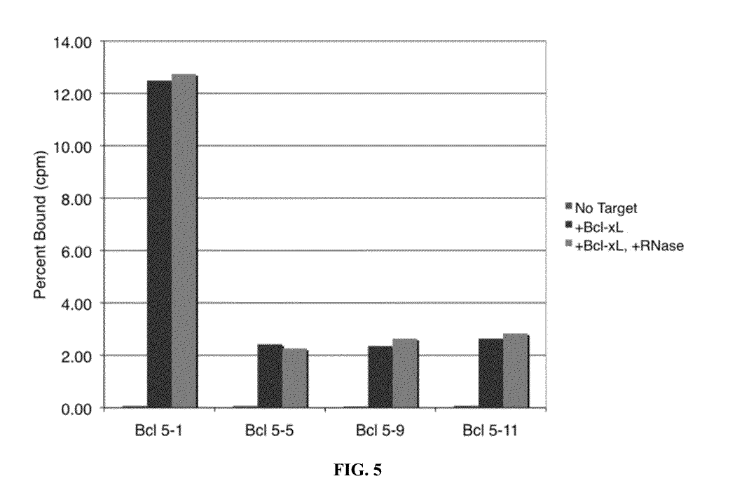

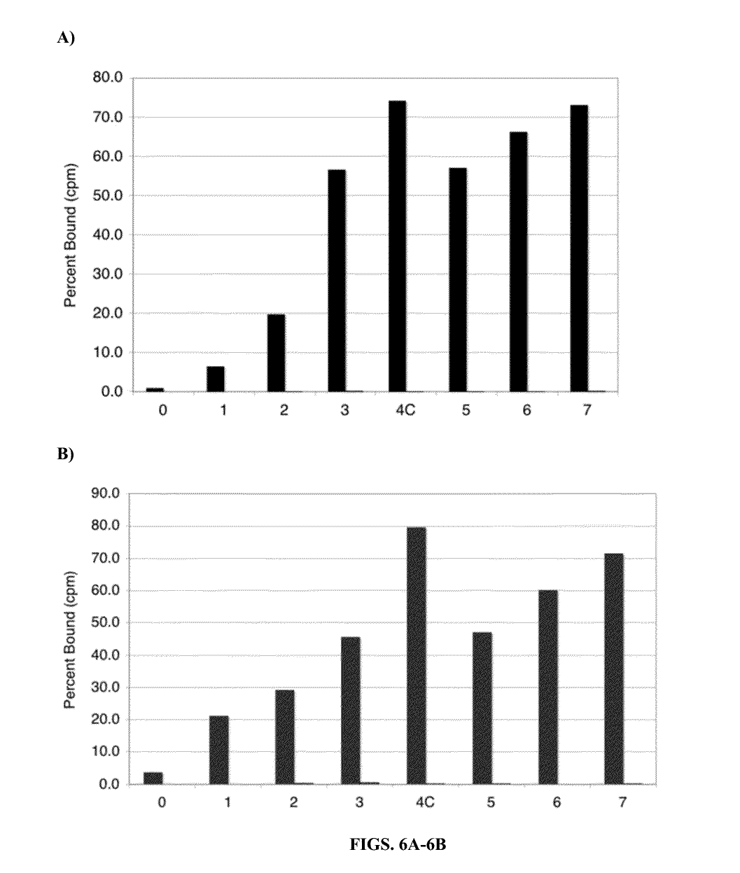

In the first selection targeting Bcl-xL (Selection 1), after five cycles of selection and amplification, significant binding over background was observed FIG. 3. Individual clone sequences from Round 5 of Selection 1 are shown in FIG. 4. The binding for several of these clone sequences is shown in FIG. 5.

The DNA from Selection 1, representing the enriched DNA library (FIG. 1) was amplified either by a primer encoding a 5′ AcuI site or a primer encoding a 3′ BpmI site. The original, random Met-X₉-4-gsgsgss library was similarly amplified with the AcuI or BpmI containing primers. These four DNA libraries were then digested with the appropriate restriction enzyme to generate AcuI or BpmI fragments, purified, and mixed with the appropriate conjugate fragment (FIG. 1). Thus, to create the N-terminally extended library, the AcuI fragment from the enriched library was mixed with the BpmI fragment of the random library and ligated with T4 DNA ligase (FIG. 1 and FIG. 2). Similarly, to create the C-terminally extended library, the BpmI fragment from the enriched library was mixed with the AcuI fragment of the random library, and ligated with T4 DNA ligase.

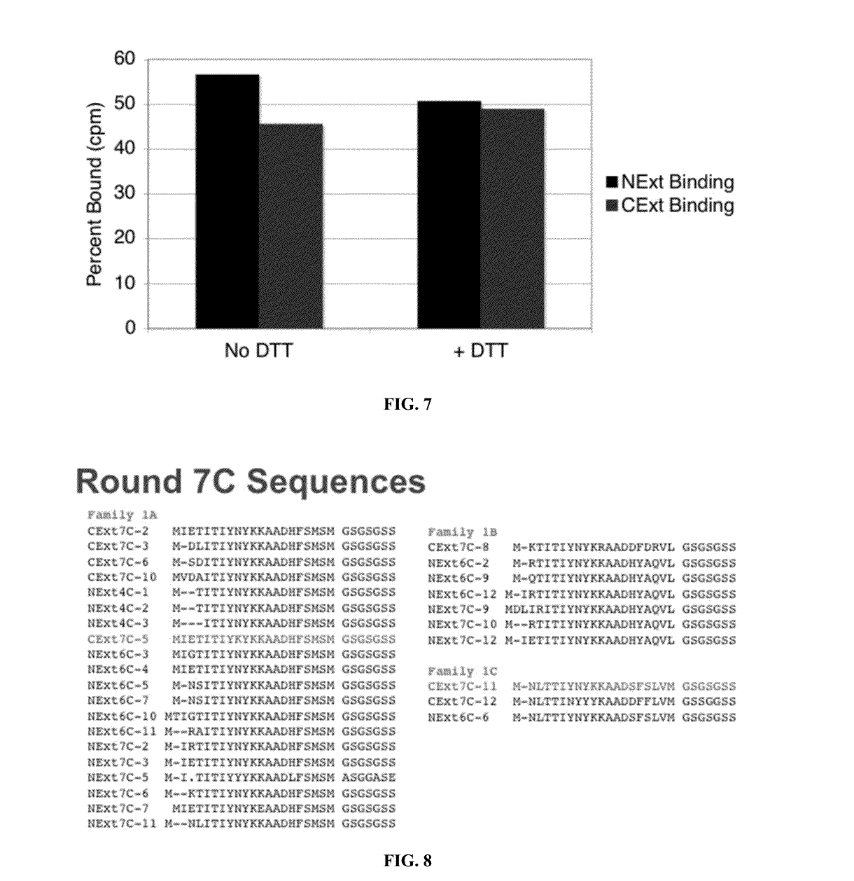

After PCR amplification, transcription, purification, the mRNA was similarly subjected to mRNA display selection. After three rounds of selection, 100-fold molar excess of free Bcl-xL without biotin (relative to immobilized Bcl-xL) was added as a competitor in order to select for peptides with very slow off rates (Boder and Wittrup (2000) Methods Enzymol 328: 430). The optimal time for the competition was determined using equations published in reference (Boder and Wittrup (1998) Biotechnol Prog 14: 55). Four additional rounds we performed for a total of 7 rounds of selection. The binding for both N- and C-terminally extended libraries are shown in FIG. 6. We then tested to see if a disulfide bond could be forming between either library and the target by adding DTT to the binding reactions. No significant difference in the presence or absence of DNA was observed (FIG. 7).

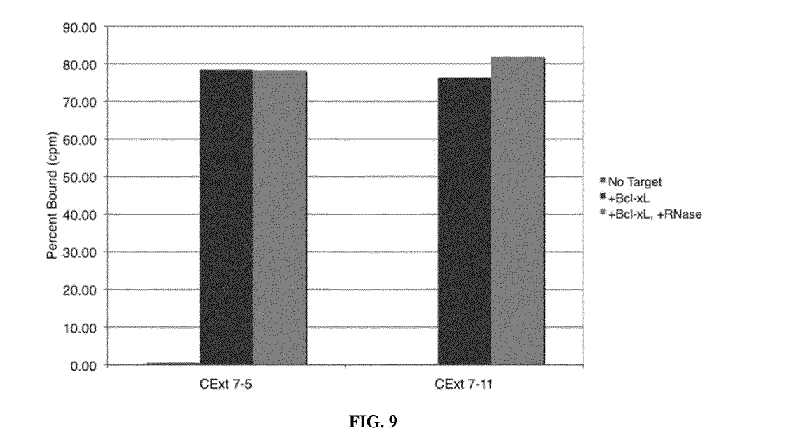

The DNA pool from Round 7 was then sequenced and the sequences shown in FIG. 8. Two clones from this round 7 were then tested for binding. FIG. 9 shows that both sequences that are radiolabeled bind well to immobilized Bclxl as a large fraction of the radioactivity is bound to the beads after washing. No binding was observed against beads without target. No significant difference in binding was observed when the attached mRNA was removed by RNase, showing that the binding was not dependent on the presence of mRNA.

Very little sequence homology is seen with known binding proteins of Bcl-xL (FIG. 10).

The off rates of clones CExt7C-5 and CExt7C11 were then tested using a radioactive off rate assay. The data show that the off rates correspond to 6×10⁻⁶ and 2×10⁻⁵ per second for CExt7-5 and CExt7-11, respectively

### Example 2

### Bcl Doped Selection Based on CExt7C-5

In a second in vitro selection, a doped library was synthesized based on the CExt7C-5 sequence. The library was synthesized such that each nucleotide was doped to be 70% of the wt and 10% of each of the remaining 3 nucleotides. For example, if the codon in the peptide was ATG, then A would be synthesized at 70% A, 10% G, 10% C, and 10% T; U would be 70% T, 10% A, 10% G and 10% T; G would be 70% G, 10% A, 10%, and 10% T, giving the ATG codon an overall 34.3% chance of being methionine.

Four rounds of mRNA display selection were performed and the resulting sequences sequenced via both Sanger and Illumina sequencing. The Sanger sequences are shown in FIG. 12. The top 20 Illumina sequences are shown in FIG. 13.

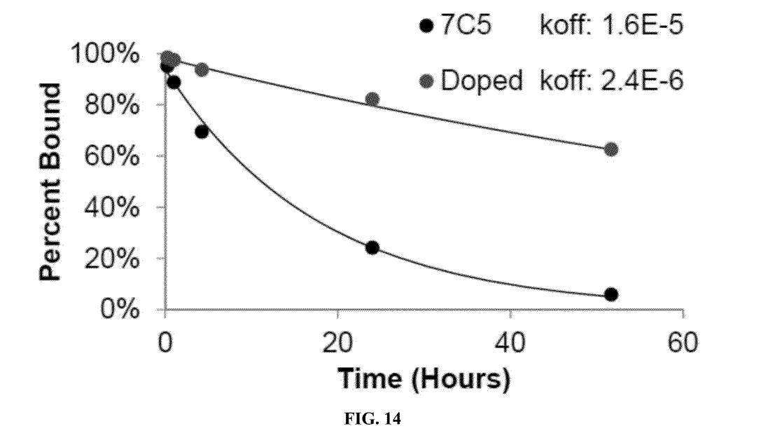

One clone, BclDopedWin 4.10 was tested to determine its off rate. The data are shown in FIG. 14. The BclDopedWin 4.10 clone has an off rate of 2.4×10⁻⁶/s.

Analysis of the Illumina sequencing data showed sequences that were functional for binding to Bcl-xL. These sequences have compositions of matter of the form:
Met at Position 1
Ile, Cys, Asp, Phe, Gly, Leu, Met, Asn, Pro, Gln, Ser, Thr, or Val at Position 2
Asp, Ala, Glu, Phe, Gly, His, Ile, Lys, Leu, Met, Asn, Pro, Gln, Arg, Ser, Thr, Val, Trp, or Tyr at Position 3
Any amino acid at Position 4
Any Amino acid at Position 5
Thr, Ala, Phe, His, Ile, Lys, Leu, Met, Gln, Arg, Ser, Val, Trp, or Tyr at Position 6
Ile, Leu, Met, Asn, Pro, Arg, or Val at Position 7
Tyr, Ala, Cys, Asp, Glu, Phe, Gly, His, Ile, Lys, Leu, Met, Asn, Gln, Arg, Ser, Thr, Val, or Trp at Position 8
Asn, Ala, Cys, Asp, Glu, Phe, His, Ile, Lys, Leu, Met, Gln, Arg, Ser, Thr, Val, or Tyr at Position 9
Tyr, Phe, Lys, Arg, Ser, or Trp at Position 10
Lys, Ile, Leu, Met, Gln, Arg, or Val at Position 11
Lys, Ala, Cys, Asp, Glu, Gly, His, Ile, Lys, Leu, Met, Asn, Gln, Arg, Ser, Thr, Val, Trp, or Tyr at Position 12
Ala, Asp, Phe, Ile, Pro, Ser, or Thr at Position 13
Ala, Leu, Met, Pro, or Ser at Position 14
Asp, Ala, or Pro at Position 15
His, Ala, Cys, Asp, Glu, Leu, Asn, Gln, Arg, Ser, Thr, Trp, or Tyr at Position 16
Phe, Ala, His, Asn, Pro, or Tyr at Position 17
Ser, Ala, Asp, Phe, Gly, His, Leu, Asn, Gln, Thr, Trp, or Tyr at Position 18
Met, Ala, Cys, Asp, Glu, Phe, Gly, His, Ile, Lys, Leu, Asn, Gln, Arg, Ser, Thr, Val, Trp, or Tyr at Position 19
Any Amino acid at Position 20
Met, Phe, His, Ile, Lys, Leu, Pro, Gln, Arg, Ser, Val, or Trp at Position 21

It is to be understood that while the invention has been described in conjunction with the above embodiments and including the attached appendix incorporated by reference herein, that the foregoing description and examples are intended to illustrate and not limit the scope of the invention. Other aspects, advantages and modifications within the scope of the invention will be apparent to those skilled in the art to which the invention pertains.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

1. A method for preparing a peptide ligand for biological activity, the method comprising:
a) obtaining an extension library comprising the step of linking an extension polynucleotide to a termini of each of a plurality of polynucleotides of target library, wherein each polynucleotide of the plurality encodes a pre-selected peptide ligand to a target peptide;
b) translating the plurality of polynucleotides of the extension library to the corresponding peptide to obtain a target peptide library;
c) screening the target peptide library for biological activity; and
d) selecting one or more of the peptide ligands of the target peptide library for the biological activity.

2. The method of claim 1, further comprising repeating steps a) through d) for each of the selected peptide ligands of step d).

3. The method of claim 2, wherein the step a) through d) are repeated more than once.

4. The method of claim 1, wherein the biological activity of step d) is selected from the group of binding affinity of the peptide ligand for the target peptide; binding specificity to the target peptide; resistance to degradation; stability; or thermostability.

5. The method of claim 1, wherein the termini of each of the plurality of polynucleotides of step a) is independently for each polynucleotide: the 5′ terminus, the 3′ terminus or both.

6. The method of claim 1, wherein the extension library is DNA or RNA.

7. The method of claim 1, wherein the plurality of polynucleotides is selected from a source library obtained from a method of the group: mRNA display; ribosome display;
phage display; TRAP display; yeast display; selex; or peptide-on-plasmids.

8. The method of claim 1, wherein the plurality of polynucleotides is selected from an mRNA display library.

9. The method of claim 1, wherein the extension library is DNA.

10. The method of claim 9, further comprising modifying the polynucleotides encoding the target peptide library to facilitate linking the random polynucleotides to the termini of each of the polynucleotides.

11. The method of claim 10, wherein the polynucleotides are modified by a method comprising PCR primer extension or restriction enzyme digestion.

12. The method of claim 1, wherein the extension polynucleotide linked to the each of the plurality of polynucleotides encoding the target peptide library comprises at least two nucleotide residues.

13. The method of claim 12, wherein between about 2 to about 150 nucleotides comprise the extension polynucleotide linked to the each of the plurality of polynucleotides encoding the target peptide library.

14. The method of claim 12 or 13, wherein the extension polynucleotide further comprises a spacer polynucleotide linked between the extension polynucleotide and the plurality of polynucleotides encoding the target peptide library.

15. The method of claim 14, wherein the extension polynucleotide encodes the peptide comprising Gly-Ser-Gly-Ser (SEQ ID NO: 1).

16. The method of claim 14, wherein the spacer polynucleotide encodes a polypeptide comprising between 1 and 100 amino acids.

17. The method of claim 1, wherein the pre-selected peptide ligand comprises an isolated naturally occurring polypeptide.

18. The method of claim 1, wherein the pre-selected peptide ligand comprises one or more unnatural amino acids.

19. The method of claim 1, wherein the plurality of polynucleotides and/or the extension polynucleotides comprise an unnatural nucleotide.

20. An isolated peptide obtainable by the method of claim 1.

21. An isolated peptide comprising an sequence shown in any one of FIG. 4, FIG. 8, FIG. 10, FIG. 11, FIG. 12 and FIG. 13.

22. The isolated peptide of claim 20, further comprising a detectable label, therapeutic agent or a cytotoxin.

23. An isolated polynucleotide encoding the peptide of claim 20.

24. A gene delivery vehicle or host cell comprising the polynucleotide of claim 23.

25. A method for targeting a cell or tissue expressing a target peptide, comprising contacting the cell or tissue with the isolated peptide of claim 20, thereby targeting the cell or tissue.

26. A method for treating or diagnosing a condition related to expression of a target peptide, comprising administering an effective amount of the isolated peptide of claim 20, to a subject in need thereof.

27. The method of claim 26, wherein the isolated peptide is administered by administration of a polynucleotide encoding the peptide.

28. A kit for performing the method of claim 1, comprising reagents to perform the methods and instructions for use.


SEQUENCE LISTING (102 sequences)

SEQ ID NO: 1 - protein, 4 aa, Artificial Sequence
  Description of Artificial Sequence Synthetic peptide
GSGS

SEQ ID NO: 2 - protein, 18 aa, Artificial Sequence
  Description of Artificial Sequence Synthetic peptide
MXXXXXXXXXXGSGSGSS

SEQ ID NO: 3 - protein, 7 aa, Artificial Sequence
  Description of Artificial Sequence Synthetic peptide
GSGSGSS

SEQ ID NO: 4 - DNA, 96 bp, Artificial Sequence
  Description of Artificial Sequence Synthetic oligonucleotide
taatacgactcactatagggacaattactatttacaattacaatgnnsnnsnnsnnsnnsnnsnnsnnsnnsntgggatctggcagtggctccagc

SEQ ID NO: 5 - DNA, 96 bp, Artificial Sequence
  Description of Artificial Sequence Synthetic oligonucleotide
taatacgactcactatagggacactgaagatttacaattacaatgnnsnnsnnsnnsnnsnnsnnsnnsnnsntgggatctggcagtggctccagc

SEQ ID NO: 6 - DNA, 48 bp, Artificial Sequence
  Description of Artificial Sequence Synthetic oligonucleotide
taatacgactcactatagggacactgaagatttacaattacaatgnns

SEQ ID NO: 7 - DNA, 45 bp, Artificial Sequence
  Description of Artificial Sequence Synthetic oligonucleotide
taatacgactcactatagggacactgaagatttacaattacaatg

SEQ ID NO: 8 - DNA, 43 bp, Artificial Sequence
  Description of Artificial Sequence Synthetic oligonucleotide
ttgtaattgtaaatcttcagtgtccctatagtgagtcgtatta

SEQ ID NO: 9 - DNA, 29 bp, Artificial Sequence
  Description of Artificial Sequence Synthetic oligonucleotide
taatacgactcactatagggacaattact

SEQ ID NO: 10 - DNA, 63 bp, Artificial Sequence
  Description of Artificial Sequence Synthetic oligonucleotide
atgnnsnnsnnsnnsnnsnnsnnsnnsnnsntgggatctggcagtggctccagcaagcagaac

SEQ ID NO: 11 - DNA, 39 bp, Artificial Sequence
  Description of Artificial Sequence Synthetic oligonucleotide
nnsnnsntgggatctggcagtggctccagcaagcagaac

SEQ ID NO: 12 - DNA, 30 bp, Artificial Sequence
  Description of Artificial Sequence Synthetic oligonucleotide
ggatctggcagtggctccagcaagcagaac

SEQ ID NO: 13 - DNA, 32 bp, Artificial Sequence
  Description of Artificial Sequence Synthetic oligonucleotide
gttctgcttgctggagccactgccagatccca

SEQ ID NO: 14 - DNA, 15 bp, Artificial Sequence
  Description of Artificial Sequence Synthetic oligonucleotide
nnsnnsntgnnsnns

SEQ ID NO: 15 - protein, 18 aa, Artificial Sequence
  Description of Artificial Sequence Synthetic peptide
MXXXXXXXXXXGSGSGSS

SEQ ID NO: 16 - protein, 15 aa, Artificial Sequence
  Description of Artificial Sequence Synthetic peptide
MKPLWPYMGSGSGSS

SEQ ID NO: 17 - protein, 18 aa, Artificial Sequence
  Description of Artificial Sequence Synthetic peptide
MWRWKMIADQLGSGSGSS

SEQ ID NO: 18 - protein, 18 aa, Artificial Sequence
  Description of Artificial Sequence Synthetic peptide
MNYDELFDDAGIWQWLQR

SEQ ID NO: 19 - protein, 18 aa, Artificial Sequence
  Description of Artificial Sequence Synthetic peptide
MNSTPPLASSMGSGSGSS

SEQ ID NO: 20 - protein, 9 aa, Artificial Sequence
  Description of Artificial Sequence Synthetic peptide
MSLTKLSTA

SEQ ID NO: 21 - protein, 18 aa, Artificial Sequence
  Description of Artificial Sequence Synthetic peptide
MGRTNLSKTYWDPAGAPT

SEQ ID NO: 22 - protein, 18 aa, Artificial Sequence
  Description of Artificial Sequence Synthetic peptide
MRLRNTSPWLWGIWVGQR

SEQ ID NO: 23 - protein, 18 aa, Artificial Sequence
  Description of Artificial Sequence Synthetic peptide
MNSIGLLPVSMGSGSGSS

SEQ ID NO: 24 - protein, 18 aa, Artificial Sequence
  Description of Artificial Sequence Synthetic peptide
MNLTELLKSYMGSGSGSS

SEQ ID NO: 25 - protein, 15 aa, Artificial Sequence
  Description of Artificial Sequence Synthetic peptide
TTLLQPYMGSGSGSS

SEQ ID NO: 26 - protein, 15 aa, Artificial Sequence
  Description of Artificial Sequence Synthetic peptide
MMLLHFSMGSGSGSS

SEQ ID NO: 27 - protein, 18 aa, Artificial Sequence
  Description of Artificial Sequence Synthetic peptide
MSSTLPQFAYMGSGSGSS

SEQ ID NO: 28 - protein, 28 aa, Artificial Sequence
  Description of Artificial Sequence Synthetic peptide
MIETITIYNYKKAADHFSMSMGSGSGSS

SEQ ID NO: 29 - protein, 27 aa, Artificial Sequence
  Description of Artificial Sequence Synthetic peptide
MDLITIYNYKKAADHFSMSMGSGSGSS

SEQ ID NO: 30 - protein, 27 aa, Artificial Sequence
  Description of Artificial Sequence Synthetic peptide
MSDITIYNYKKAADHFSMSMGSGSGSS

SEQ ID NO: 31 - protein, 28 aa, Artificial Sequence
  Description of Artificial Sequence Synthetic peptide
MVDAITIYNYKKAADHFSMSMGSGSGSS

SEQ ID NO: 32 - protein, 26 aa, Artificial Sequence
  Description of Artificial Sequence Synthetic peptide
MTITIYNYKKAADHFSMSMGSGSGSS

SEQ ID NO: 33 - protein, 26 aa, Artificial Sequence
  Description of Artificial Sequence Synthetic peptide
MTITIYNYKKAADHFSMSMGSGSGSS

SEQ ID NO: 34 - protein, 25 aa, Artificial Sequence
  Description of Artificial Sequence Synthetic peptide
MITIYNYKKAADHFSMSMGSGSGSS

SEQ ID NO: 35 - protein, 28 aa, Artificial Sequence
  Description of Artificial Sequence Synthetic peptide
MIETITIYKYKKAADHFSMSMGSGSGSS

SEQ ID NO: 36 - protein, 28 aa, Artificial Sequence
  Description of Artificial Sequence Synthetic peptide
MIGTITIYNYKKAADHFSMSMGSGSGSS

SEQ ID NO: 37 - protein, 28 aa, Artificial Sequence
  Description of Artificial Sequence Synthetic peptide
MIETITIYNYKKAADHFSMSMGSGSGSS

SEQ ID NO: 38 - protein, 27 aa, Artificial Sequence
  Description of Artificial Sequence Synthetic peptide
MNSITIYNYKKAADHFSMSMGSGSGSS

SEQ ID NO: 39 - protein, 27 aa, Artificial Sequence
  Description of Artificial Sequence Synthetic peptide
MNSITIYNYKKAADHFSMSMGSGSGSS

SEQ ID NO: 40 - protein, 29 aa, Artificial Sequence
  Description of Artificial Sequence Synthetic peptide
MTIGTITIYNYKKAADHFSMSMGSGSGSS

SEQ ID NO: 41 - protein, 27 aa, Artificial Sequence
  Description of Artificial Sequence Synthetic peptide
MRAITIYNYKKAADHFSMSMGSGSGSS

SEQ ID NO: 42 - protein, 28 aa, Artificial Sequence
  Description of Artificial Sequence Synthetic peptide
MIRTITIYNYKKAADHFSMSMGSGSGSS

SEQ ID NO: 43 - protein, 28 aa, Artificial Sequence
  Description of Artificial Sequence Synthetic peptide
MIETITIYNYKKAADHFSMSMGSGSGSS

SEQ ID NO: 44 - protein, 27 aa, Artificial Sequence
  Description of Artificial Sequence Synthetic peptide
MITITIYYYKKAADLFSMSMASGGASE

SEQ ID NO: 45 - protein, 27 aa, Artificial Sequence
  Description of Artificial Sequence Synthetic peptide
MKTITIYNYKKAADHFSMSMGSGSGSS

SEQ ID NO: 46 - protein, 28 aa, Artificial Sequence
  Description of Artificial Sequence Synthetic peptide
MIETITIYNYKEAADHFSMSMGSGSGSS

SEQ ID NO: 47 - protein, 27 aa, Artificial Sequence
  Description of Artificial Sequence Synthetic peptide
MNLITIYNYKKAADHFSMSMGSGSGSS

SEQ ID NO: 48 - protein, 27 aa, Artificial Sequence
  Description of Artificial Sequence Synthetic peptide
MKTITIYNYKRAADDFDRVLGSGSGSS

SEQ ID NO: 49 - protein, 27 aa, Artificial Sequence
  Description of Artificial Sequence Synthetic peptide
MRTITIYNYKKAADHYAQVLGSGSGSS

SEQ ID NO: 50 - protein, 27 aa, Artificial Sequence
  Description of Artificial Sequence Synthetic peptide
MQTITIYNYKKAADHYAQVLGSGSGSS

SEQ ID NO: 51 - protein, 28 aa, Artificial Sequence
  Description of Artificial Sequence Synthetic peptide
MIRTITIYNYKKAADHYAQVLGSGSGSS

SEQ ID NO: 52 - protein, 29 aa, Artificial Sequence
  Description of Artificial Sequence Synthetic peptide
MDLIRITIYNYKKAADHYAQVLGSGSGSS

SEQ ID NO: 53 - protein, 27 aa, Artificial Sequence
  Description of Artificial Sequence Synthetic peptide
MRTITIYNYKKAADHYAQVLGSGSGSS

SEQ ID NO: 54 - protein, 28 aa, Artificial Sequence
  Description of Artificial Sequence Synthetic peptide
MIETITIYNYKKAADHYAQVLGSGSGSS

SEQ ID NO: 55 - protein, 27 aa, Artificial Sequence
  Description of Artificial Sequence Synthetic peptide
MNLTTIYNYKKAADSFSLVMGSGSGSS

SEQ ID NO: 56 - protein, 27 aa, Artificial Sequence
  Description of Artificial Sequence Synthetic peptide
MNLTTINYYYKAADDFFLVMGSSGGSS

SEQ ID NO: 57 - protein, 27 aa, Artificial Sequence
  Description of Artificial Sequence Synthetic peptide
MNLTTIYNYKKAADSFSLVMGSGSGSS

SEQ ID NO: 58 - protein, 16 aa, Unknown
  Description of Unknown Bak peptide sequence
GQVGRQLAIIGDDINR

SEQ ID NO: 59 - protein, 16 aa, Unknown
  Description of Unknown Bax peptide sequence
KKLSECLKRIGDELDS

SEQ ID NO: 60 - protein, 16 aa, Unknown
  Description of Unknown Bok peptide sequence
AEVCAVLLRLGDELEM

SEQ ID NO: 61 - protein, 16 aa, Unknown
  Description of Unknown Bad peptide sequence
AQRYGELRRMSDEFVD

SEQ ID NO: 62 - protein, 16 aa, Unknown
  Description of Unknown Puma peptide sequence
REIGAQLRRMADDLNA

SEQ ID NO: 63 - protein, 16 aa, Unknown
  Description of Unknown Noxa peptide sequence
VECATQLRRFGDKLNF

SEQ ID NO: 64 - protein, 16 aa, Unknown
  Description of Unknown Bik peptide sequence
SDALARLACIGDEMDV

SEQ ID NO: 65 - protein, 16 aa, Unknown
  Description of Unknown Bid peptide sequence
IRNIAHLAQVGDSMDR

SEQ ID NO: 66 - protein, 16 aa, Unknown
  Description of Unknown Hrk peptide sequence
AQLTARLKALGDELHQ

SEQ ID NO: 67 - protein, 16 aa, Unknown
  Description of Unknown Bim peptide sequence
EIWIAELRRIGDEFNA

SEQ ID NO: 68 - protein, 16 aa, Unknown
  Description of Unknown A10 peptide sequence
FPRWKLLAHWADRWWF

SEQ ID NO: 69 - protein, 28 aa, Artificial Sequence
  Description of Artificial Sequence Synthetic peptide
MIETITIYNYKKAADHFSMSMGSGSGSS

SEQ ID NO: 70 - protein, 28 aa, Artificial Sequence
  Description of Artificial Sequence Synthetic peptide
MIETITIYKYKKAADHFSMSMGSGSGSS

SEQ ID NO: 71 - protein, 27 aa, Artificial Sequence
  Description of Artificial Sequence Synthetic peptide
MNLTTIYNYKKAADSFSLVMGSGSGSS

SEQ ID NO: 72 - protein, 28 aa, Artificial Sequence
  Description of Artificial Sequence Synthetic peptide
MIETITIYNYKKAADHFSMSMGSGSGSS

SEQ ID NO: 73 - protein, 28 aa, Artificial Sequence
  Description of Artificial Sequence Synthetic peptide
MIDTLTIYNYKKAADHFAQSMGSGSGSS

SEQ ID NO: 74 - protein, 28 aa, Artificial Sequence
  Description of Artificial Sequence Synthetic peptide
MIDTITLYNYRKAADHFSMTLGSGSGSS

SEQ ID NO: 75 - protein, 28 aa, Artificial Sequence
  Description of Artificial Sequence Synthetic peptide
MIESITIYNYKKAADRYALSKGSGSGSS

SEQ ID NO: 76 - protein, 28 aa, Artificial Sequence
  Description of Artificial Sequence Synthetic peptide
MIELISIINYKKAADHFSMSMGSGSGSS

SEQ ID NO: 77 - protein, 28 aa, Artificial Sequence
  Description of Artificial Sequence Synthetic peptide
MIAISTIYNYKKAADHYAMTKGSGSGSS

SEQ ID NO: 78 - protein, 28 aa, Artificial Sequence
  Description of Artificial Sequence Synthetic peptide
MIKSITIYKYKQAADHFAMSMGSGSGSS

SEQ ID NO: 79 - protein, 28 aa, Artificial Sequence
  Description of Artificial Sequence Synthetic peptide
MIDYITIYNYKNAADHYSLFMGSGSGSS

SEQ ID NO: 80 - protein, 28 aa, Artificial Sequence
  Description of Artificial Sequence Synthetic peptide
MIERIAIYNYKKAADHFAMSIGSGSGSS

SEQ ID NO: 81 - protein, 28 aa, Artificial Sequence
  Description of Artificial Sequence Synthetic peptide
MIETTTIYNYKKAADHYSMSKGSGSGSS

SEQ ID NO: 82 - protein, 28 aa, Artificial Sequence
  Description of Artificial Sequence Synthetic peptide
MIAISTIYNYKKAADHYAMTKGSGSGSS

SEQ ID NO: 83 - protein, 25 aa, Artificial Sequence
  Description of Artificial Sequence Synthetic peptide
MIDTITIYNYKKAADHFSMSMGSGS

SEQ ID NO: 84 - protein, 25 aa, Artificial Sequence
  Description of Artificial Sequence Synthetic peptide
MIDPIVIYEYKKAADRFSILMGSGS

SEQ ID NO: 85 - protein, 25 aa, Artificial Sequence
  Description of Artificial Sequence Synthetic peptide
MIETITIYNYKKAADHFSMSMGSGS

SEQ ID NO: 86 - protein, 25 aa, Artificial Sequence
  Description of Artificial Sequence Synthetic peptide
MIDAITIYNYKKAADHFSMSMGSGS

SEQ ID NO: 87 - protein, 25 aa, Artificial Sequence
  Description of Artificial Sequence Synthetic peptide
MIATITIYNYKKAADHFSMSMGSGS

SEQ ID NO: 88 - protein, 25 aa, Artificial Sequence
  Description of Artificial Sequence Synthetic peptide
MIDTITIYNYKKAADHFSISMGSGS

SEQ ID NO: 89 - protein, 25 aa, Artificial Sequence
  Description of Artificial Sequence Synthetic peptide
MIEAITIYNYKKAADHFSMSMGSGS

SEQ ID NO: 90 - protein, 25 aa, Artificial Sequence
  Description of Artificial Sequence Synthetic peptide
MIDTLTIYNYKKAADHFSMSMGSGS

SEQ ID NO: 91 - protein, 25 aa, Artificial Sequence
  Description of Artificial Sequence Synthetic peptide
MIDTITIYNYKKAADHFSLSMGSGS

SEQ ID NO: 92 - protein, 25 aa, Artificial Sequence
  Description of Artificial Sequence Synthetic peptide
MIETSVIYNYKRAADHFYMSMGSGS

SEQ ID NO: 93 - protein, 25 aa, Artificial Sequence
  Description of Artificial Sequence Synthetic peptide
MIEPITIYNYKKAADHFSMSMGSGS

SEQ ID NO: 94 - protein, 25 aa, Artificial Sequence
  Description of Artificial Sequence Synthetic peptide
MIDTKTLYNYKRAADHFAMSMGSGS

SEQ ID NO: 95 - protein, 25 aa, Artificial Sequence
  Description of Artificial Sequence Synthetic peptide
MIDTITILNYKRAADHFAMSIGSGS

SEQ ID NO: 96 - protein, 25 aa, Artificial Sequence
  Description of Artificial Sequence Synthetic peptide
MIDAITIYNYKKAADHFSISMGSGS

SEQ ID NO: 97 - protein, 25 aa, Artificial Sequence
  Description of Artificial Sequence Synthetic peptide
MIDAITIYNYKRAADHFSMSIGSGS

SEQ ID NO: 98 - protein, 25 aa, Artificial Sequence
  Description of Artificial Sequence Synthetic peptide
MIETSTIFAYKAAADHFNLSMGSGS

SEQ ID NO: 99 - protein, 25 aa, Artificial Sequence
  Description of Artificial Sequence Synthetic peptide
MLSTTVIDNFKKAADRFNIYLGSGS

SEQ ID NO: 100 - protein, 25 aa, Artificial Sequence
  Description of Artificial Sequence Synthetic peptide
MIEAITIYNYKKAADHFSISMGSGS

SEQ ID NO: 101 - protein, 25 aa, Artificial Sequence
  Description of Artificial Sequence Synthetic peptide
MIATITIYNYKKAADHFSISMGSGS

SEQ ID NO: 102 - protein, 25 aa, Artificial Sequence
  Description of Artificial Sequence Synthetic peptide
MIDTITIYNYKKAADHFSMTMGSGS